(19) United States
(12) Patent Application Publication
(10) Pub. No.: US 2004/0106160 A1
(43) Pub. Date: Jun. 3, 2004

(54) Screening assay for antagonists of human leukocyte receptors

(76) Inventors: Zhenyi Xu, Lund (SE); K M Erik Michaelsson, Asa (SE); Leif Petersson, Malmo (SE); Paul Sorensen, Kopenhamn (SE)

Correspondence Address: BURNS DOANE SWECKER & MATHIS L L P, POST OFFICE BOX 1404, ALEXANDRIA, VA, 22313-1404, US

(21) Appl. No.: 10/432,726

(22) PCT Filed: Dec. 20, 2001
(86) PCT No.: PCT/SE01/02841

(30) Foreign Application Priority Data
Dec. 22, 2000 (SE) 0004781-1

(51) Int. Cl. C12Q 1/68; G01N 33/567

(52) U.S. Cl. 435/7.21; 435/6

(57) ABSTRACT

A method of screening compounds for their ability of inhibiting ligand-induced co-stimulatory receptor internalisation pathways in immune competent human cells is described. The immune competent human cells are incubated at conditions capable of inducing co-stimulatory receptor internalisation in the presence of at least one test compound and the suppression of the ligand-induced co-stimulatory receptor internalisation is determined. There is also described a kit for use in such a method, as well as an immunoregulatory drug capable of blocking down-modulation of a ligand-induced receptor.

### FIELD OF THE INVENTION

The present invention relates to a method of screening compounds for their ability of inhibiting ligand-induced co-stimulatory receptor internalisation pathways in immune competent human cells, a kit for use in screening said compounds and an immuno-regulatory drug capable of blocking down-modulation of a ligand-induced receptor thus preventing ligand-induced receptor internalisation (LIRI).

### BACKGROUND OF THE INVENTION

The activation of mature T lymphocytes requires antigen recognition and secondary signals collectively called co-stimulation (1). It is now believed that antigen recognition through the T cell receptor (TCR) alone can not activate T cells, but rather induces a state of unresponsiveness known as energy. The engagement of co-stimulatory pathways is necessary for optimising T cell activation. The best characterised co-stimulatory receptor expressed on a resting T cell is CD28. Interaction of CD28 with its ligands, CD80 and CD86, plays a crucial role in augmenting and sustaining a T cell response initiated through the TCR engagement (2). Co-stimulation through CD2/LFA3, CD40L/CD40, LFA-1/ICAM pathways (3) have also been documented. More recently it was discovered that 4-1BB and ICOS are functioning as co-stimulation molecules (4).

Internalisation of receptors has long been a fascinating subject for biologists (5). Although underlying mechanisms have not been fully elucidated tremendous knowledge has been accumulated. Internalisation of co-stimulatory receptors after engagement with specific mAbs have been noticed. More recently a natural ligand-induced receptor internalisation has been reported for CD28/B7, CD40/CD154 pathways (6).

Since the co-stimulatory signals are pivotal in determining recognition of antigen, either by T cell to T cell activation or by allergy, the role of the co-stimulation in the development of autoimmune responses is obvious. Co-stimulatory signals provide a second signal, which determines the outcome of TCR engagement since they augment T cell proliferation and the functions of effector cell, such as cytokine production and cytolysis. It has been suggested that the absence of co-stimulators on resting tissue antigen presenting cells (APCs) could serve to induce and maintain T cell tolerance to self-antigens and that aberrant expression of co-stimulators on APCs could stimulate self-reactive T cells, resulting in autoimmunity. In fact it has been demonstrated that blockage of co-stimulation ameliorated autoimmune responses in several animal disease models. Evidence has also come from “knock-out” mice whose genes for co-stimulatory molecules have been genetically deleted, showing that the mice did not develop, or only mildly developed, autoimmune diseases.

Due to the involvement of co-stimulation in inflammation and autoimmune diseases in human, numerous studies have been conducted trying to ameliorate the abnormal responses by blocking co-stimulatory pathways. Various large biomolecules, such as monoclonal antibodies, either intact or genetically manipulated, against either the receptors or the ligands, immunoglobulins binding to receptors, or soluble ligands, have been demonstrated to be effective in vitro on inhibiting co-stimulation, and promising results have also been obtained from in vivo studies (1).

Later efforts have been made in identifying small molecular compounds which are able to down-modulate co-stimulation with a focus on specifically inhibiting binding between a co-stimulatory receptor and its ligand. Methods have been used, which are based on isolated targets or cell interactions characterised by interactions through complex molecular assemblies of cell surface receptors, for identifying such compounds.

In previously known methods the set-up is typically, at the best, only partially cell based. In a scintillation proximity assay (SPA), for instance, the receptor or the membrane in which it resides is immobilised onto or captured by beads containing a tracer and the appropriate ligand is radiolabeled. When the receptor binds to the tracer it brings the radioisotope close enough to the bead to stimulate the scintillant to emit light. By contrast, if an unlabeled ligand or competing drug replaces the tracer in the receptor binding site, less radioactivity is associated with the bead and consequently less light is emitted. Thus, at equilibrium, the presence of molecules that are able to compete with the radiotracer for the receptor may be detected.

However, these previous methods have the disadvantage of not being able to predict the efficacy and molecular interactions of the tested compounds in vivo. Furthermore, they are time-consuming since they require expression steps and protein purification. Generally, the methods also include long co-incubation times with the tested compound, which can lead to great background “noise” in the results since it is hard to distinguish the results of the toxicity of the test compound from its desirable effects.

Abbreviation list

Throughout the rest of the text the following abbreviations are used:

| | |
|---|---|
| ACAS | activated cell analysis and sorting |
| CHO | Chinese hamster ovary |
| CD | cluster of differentiation |
| FCS | fetal calf serum |
| FACS | fluorescence activated cell sorter |
| FTIC | fluorescein isothiocyanate |
| HLA | human leukocyte antigen |
| ICAM | intercellular adhesion molecule |
| ICOS | inducible co-stimulator |
| LFA | leukocyte function antigen |
| LIRI | ligand-induced receptor internalisation |
| PBMC | peripheral blood mononuclear cells |
| PFA | paraformaldehyde |
| PE | phycoerythrin |
| SEA | staphylococcal enterotoxin A |
| SPA | scintillation proximity assay |

### SUMMARY OF THE INVENTION

The present invention relates in a first aspect to a method of screening compounds for their ability of inhibiting ligand-induced co-stimulatory receptor internalisation pathways in immune competent human cells. Said immune competent cells are incubated at conditions capable of inducing co-stimulatory receptor internalisation in the presence of at least one test compound, and then the suppression of the ligand-induced co-stimulatory receptor internalisation is determined.

In one embodiment the immune competent cells of the method are leukocytes. In a further embodiment the leukocytes are lymphocytes. In still another embodiment the lymphocytes are T-cells. In yet another embodiment the T-cells are Jurkat cells.

In another embodiment said leukocytes are antigen presenting cells. In yet another embodiment the antigen presenting cells are B-cells.

In a further embodiment, said conditions of the method imply culturing the immune competent cells with Chinese hamster ovarian (CHO) cells transfected with a DNA, which codes for at least one human ligand. In a still further embodiment the CHO cells are transfected with a DNA encoding at least one human ligand or receptor chosen from the group comprising ICAM, CD54(LFA3), CD40, CD80, CD86, CD154(CD40L).

In another embodiment of the method the test compound is a low molecular weight compound, which preferably has a molecular weight of up to about 500.

In still another embodiment of the method, the determination of the suppression is made by flow cytometry or confocal microscopy analysis of the cells.

The method is advantageously automated for high content screening (HCS) or medium through-put screening (MTS).

In another embodiment of the method, said pathways are chosen from the group of receptor-ligand pairs comprising CD40/CD154(CD40L); CD2/LFA3; CD28/CD80, CD86; and CD11l/ICAM.

In another aspect the invention relates to a kit for use in screening compounds for their ability of inhibiting ligand-induced co-stimulatory receptor internalisation in immune competent human cells, comprising means for culturing immune competent human cells, means for inducing co-stimulatory receptor internalisation, means for incubating the immune competent human cells with at least one test compound, means for marking the receptors and means for determining suppression of the ligand-induced co-stimulatory receptor internalisation.

In one embodiment of said kit, a conjugate with an isotope or a fluorescent protein is used for marking the receptors.

In another embodiment of said kit, flow cytometry or confocal microscopy is used for determining suppression of the ligand-induced co-stimulatory receptor internalisation.

In still another aspect the invention relates to an immuno-regulatory drug, capable of blocking down-modulation of a ligand-induced receptor thus preventing ligand-induced receptor internalisation.

In one embodiment the immuno-regulatory drug is a low molecular weight compound. In a further embodiment the compound has a molecular weight of up to about 500.

### LEGENDS OF FIGURES

FIG. 1. Human CD80 and CD86 induce CD28 receptor down-regulation.

FIG. 2. Dose-dependency and time course of CD28 down-modulation induced by CD80. A: Jurkat cells were cultured with different number of CHO/CD80 cells. B: Jurkat cells were incubated with CHO/CD80 at a ratio of 5:1 for different times.

FIG. 3. LFA3 (CD58) down-modulates CD2 expression on T cells.

FIG. 4. Human CD40L and CD40 down-regulate each other. A: Human B cell line Ramos 2G6 4C10 cell were co-incubated with CHO transfectants. B: CD40L⁺-Jurkat cells were incubated with CHO/DR or CHO/DR/CD40 cells for different times.

FIG. 5. LFA-3 and CD80 induces CD2 and CD28 receptor down-modulation in human PBMC.

FIG. 6. Human ICAM1 (CD54) down-modulates CD11α(LFA-1) and αICAM1 mAb blocks the effect.

FIG. 7. Pre-treatment of either CD80 on CHO/CD80 or CD28 on Jurkat with the mAbs blocks CD80-induced CD28 down-modulation. A: CHO/CD80 transfectants were incubated with αCD80 mAb or control mAb B: Jurkat cells were incubated with αCD28 mAb.

FIG. 8. Pretreatment of CHO/CD40 or Raji cells with αCD40 mAb.

FIG. 9. mAbs that block co-stimulatory receptor down-modulation also block the T cell activation. A: Jurkat cells pretreated with different concentrations of αCD28 mAb or control mAb co-cultured with CHO/DR/CD80 transfectants and superantigen SEE. B: Jurkat cells pretreated with αCD2 mAb or control mAb co-cultured with CHO/DR/LFA3 transfectants.

FIG. 10. Intracellular staining demonstrates that receptors are internalised after interacting with the ligands. A: Jurkat cells were cultured with either CHO/LFA3 or CHO/CD40. B: Culture conditions and intracellular staining, procedures are the same as in A.

FIG. 11. Substance L specifically inhibits ligand A-induced receptor A down-modulation.

FIG. 12. Induction of CTLA-4 on human T cells. Human PBMC were activated with SEE (5 nM) for 72 hours and phenotypically analyzed by FACS.

FIG. 13. CD80 down-modulates CTLA-4 expression on SEE-activated human PBMC. Human PBMC which had been activated with SEE (5 nM) for 72 hours were mixed with CHO/CD80 and incubated for 30 minutes. The expression of CTLA-4 was analyzed by FACS.

### DETAILED DESCRIPTION OF THE INVENTION

Co-stimulation plays a crucial role in both human T and B lymphocyte activation. Blockage of the co-stimulatory pathways may for example ameliorate autoimmune diseases, which are characterised by abnormal T cell and B cell activation.

Observations were made which confirmed that interaction between the ligand and the receptor inevitably results in internalisation of the receptor, in a specific and highly sensitive manner. Addition of competitive binders, e.g. monoclonal antibodies against the receptor or the ligand, prevented the ligand-induced receptor internalisation (LIRI).

The present invention comprises a new screening method for discovery of antagonists of co-stimulatory receptors on human lymphocytes. The method is based e.g. on flow cytometry analysis and a large number of compounds may be screened by this assay. It has for example been found that by this method one low molecular weight compound, substance L, which is a pteridine derivative with a molecular weight of 321.39, specifically blocks a ligand-induced receptor down-modulation.

The compounds found by this assay, which specifically inhibit LIRI, are considered as candidates for immuno-regulatory drugs.

It has now been shown that small molecular weight compounds, exemplified by substance L, specifically inhibit LIRI. It is the first observation that small molecule weight compounds are able to block the natural ligand-induced membrane-bound receptor internalisation. This demonstrates the availability of the method for screening test compounds.

The method according to the invention uses intact, living cells instead of isolated targets or cell preparations, which are the usual ways of screening compounds. By using the method according to the invention the complexity of cell-cell interactions, characterised by interactions through complex molecular assemblies of cell surface receptors, can be considered. The efficacy of the compounds tested can be predicted by measuring biological behaviour and function.

Further, the molecular interactions can be evaluated within the natural context of the cell, toxicity and non-specific effects can be identified and drug effects on selective cell types can be distinguished. The whole cell assay obviate the protein purification and expression steps otherwise required.

The short co-incubation time of the method according to the invention also reduces the influence of possible toxicity of the test of compounds, which otherwise can lead to background “noise” in the results.

Experimental part

Materials and methods

Cells

The human Jurkat T leukemia cell line was cultured in RPMI 1640 supplemented with 2 mM glutamine and 10% FCS. The Jurkat sublines, CD40L⁺Jurkat (>95% positive) and CD40L⁻ Jurkat (>99% negative), were established by FACS sorting and cultured under same conditions.

Human SEA-maintained T cell line was established by stimulating human PBMC with SEA (5 nM) and SEA-supplemented media was changed every 5 days.

Ramos 2G6 4C10, a human B cell line, was cultured in RPMI 1640 supplemented with 10% FCS.

Chinese hamster ovarian (CHO) cells were transfected with cDNA encoding human HLA-DR₄, ICAM-1, CD80, CD86, LFA-3(CD58), CD40 and CD40L(CD154) and the cell lines were maintained in the selection media. The transfectant cell lines used in this study were: CHO-DR₄, CHO-CD28, CHO-LFA3, CHO-CD40, CHO-CD40L, CHO-ICAM1, CHO-DR₄-CD80-LFA3.

Reagents

The following monoclonal antibodies (mAbs) were used: αCD2-FITC (30054X, PharMingen), (CD3-FITC (30140X, PharMingen), αCD11a-PE (30425X, PharMingen), αCD28 (clone CD28.2, Immunotech), αCD28-FITC (33744X, PharMingen), αCD28-PE (348047, Becton Dickinson), αCD40L (33585X, PharMingen), αCD40-FITC (22074X, PharMingen), αCD80-PE (340294, Becton Dickinson), αCD86-PE (33435X, PharMingen), αLFA₃-FITC (AHS5808, BioSource), αICAM-PE (31625X, PharMingen), αHLA-DR-PE (347367, Becton Dickinson), goat-αmIgG1-FITC (M32101, CALTAG) and rabbit-αmIg-FITC (F0261, DAKO).

Substance L, a pteridine derivative with a molecular weight of 321.39, was synthesised.

Cell Cultures

Jurkat cells (1×10⁶/ml) were cultured with CHO transfectants (2×10⁵/ml) in the 12×75 mm culture tubes (Falcon 2052) for different time at 37° C. and an atmosphere of 5% CO₂. To observe effects of monoclonal antibodies or substances on LIRI, CHO transfectants or Jurkat cells were incubated with corresponding mAbs or substances for 30 minutes at 37° C. and then cultured with Jurkat cells or CHO transfectants.

Flow Cytometer and adherent cell analysing with sort (ACAS) Assay

After culture the cells were washed once with phosphate-buffered saline (PBS) and stained with fluorescence-conjugated antibodies at a cell density of 0.5×10⁶/50 μl for 30 minutes at 4° C. The cells were washed twice and resuspended in PBS. Before FACS running the cells were kept at 4° C. The cell samples were run with FACSort (Becton Dickinson) and analysed with the software Cell Quest (Becton Dickinson). Part of the cell samples was also analysed with the interactive laser cytometer (ACAS 570, Ameridian).

### EXAMPLE 1

### CD80 and CD86 Induced CD28 Down-modulation

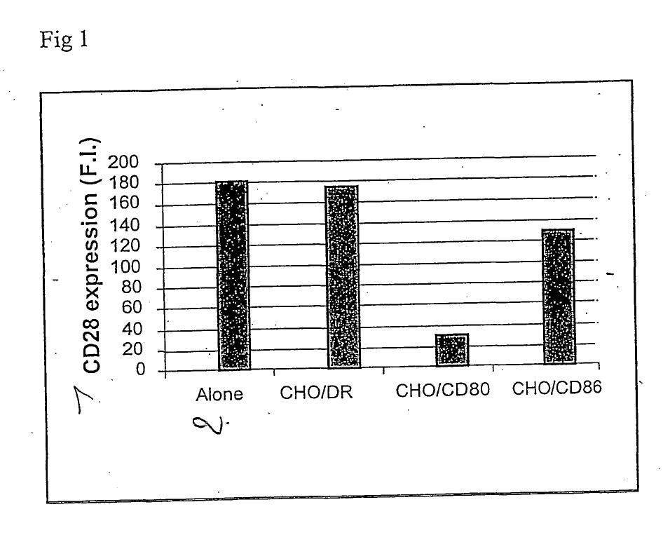

Human T cells express CD28 receptors on the cell surface and binding of the receptor with the ligands, CD80 or CD86, constitutes a vital co-stimulation signal for T cell activation. Human T cell line, Jurkat cells, and Chinese hamster ovarian (CHO) cells transfected with the ligands were applied to observe the fate of the receptor. After co-incubation for 30 minutes, the cells were washed and stained with αCD28 mAb conjugated with PE. FACS analysis results show that CD80,strongly induces CD28 down-modulation and CD86 has the less capacity. A control cell line CHO-DR, does not interfere with the receptor expression (FIG. 1).

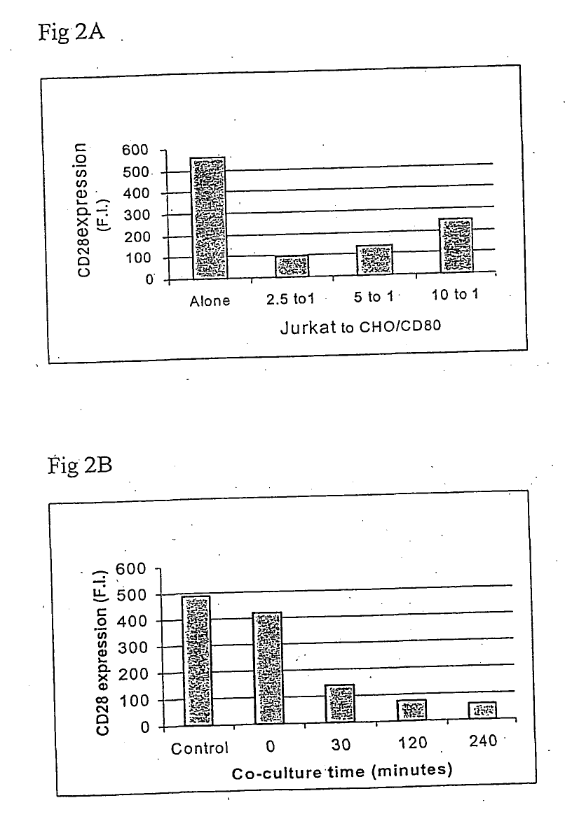

In order to investigate whether down-modulation of CD28 correlated with the number of ligands in the incubation system, Jurkat cells (0,5×10⁶/ml) were incubated for 30 minutes with various number of CHO/CD80 cells so that the Jurkat cells to CHO/CD80 ratios were 2.5−10 to 1. The results clearly show more CD80 ligands and less CD28 receptors on the cell surface (FIG. 2A). The control cell line (CHO/DR) never down-modulates CD28 expression even at higher cell numbers.

Another set of experiments show that CD80 quickly induces CD28 down-modulation. Jurkat cells were incubated with CHO/CD80 at a ratio of 5:1 for different times. After incubation the cells were harvested and analysed with FACS. After 30 minutes of co-incubation about 70% of the surface CD28 receptors were internalised. Even when the two types of cells were mixed and immediately centrifuged and stained (time “0”), around 15% of the receptors were already internalised (FIG. 2B). As far as is known this is the first time it has been documented that CD28 is so quickly internalised by binding to the natural ligands, CD80.

### EXAMPLE 2

### LFA3(CD58) Induced CD2 Down-modulation

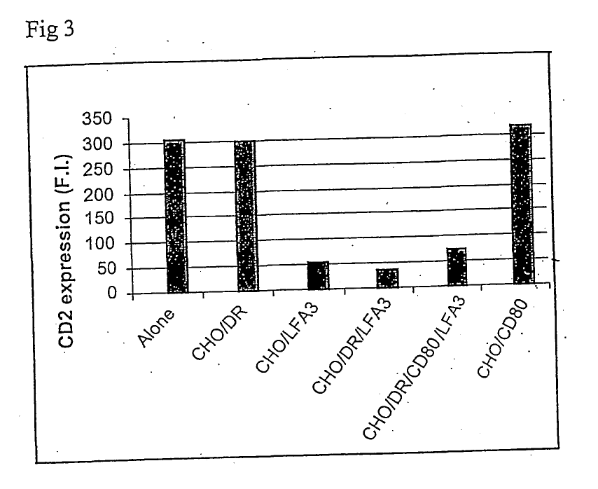

Interaction between CD2 on T cells and LFA3 on APC cells plays an important role on T-APC cell to cell adhesion and T cell co-stimulation. In this experiment we showed that LFA3 down-modulates CD2 expression on the receptor level. Jurkat cells were co-incubated with CHO cells transfected with different human molecules for 30 minutes. After incubation the cells were harvested, stained with αCD2-PE and expression of CD2 was analysed with FACS. CHO transfectants, when only transfected with LFA3, induced CD2 down modulation (FIG. 3). Other human molecules transfected into CHO cells did not interfere with the CD2 expression. The CHO/LFA3 transfectants did not interfere with the CD28 or other T cell receptor expression. LFA3 usually induces more dramatic CD2 down-modulation than CD80 does on CD28 expression. As we have searched in the literature, it is the first observation on CD2 internalisation induced by its natural ligand LFA3.

### EXAMPLE 3

### CD40 and CD40L Induce each others Down-modulation

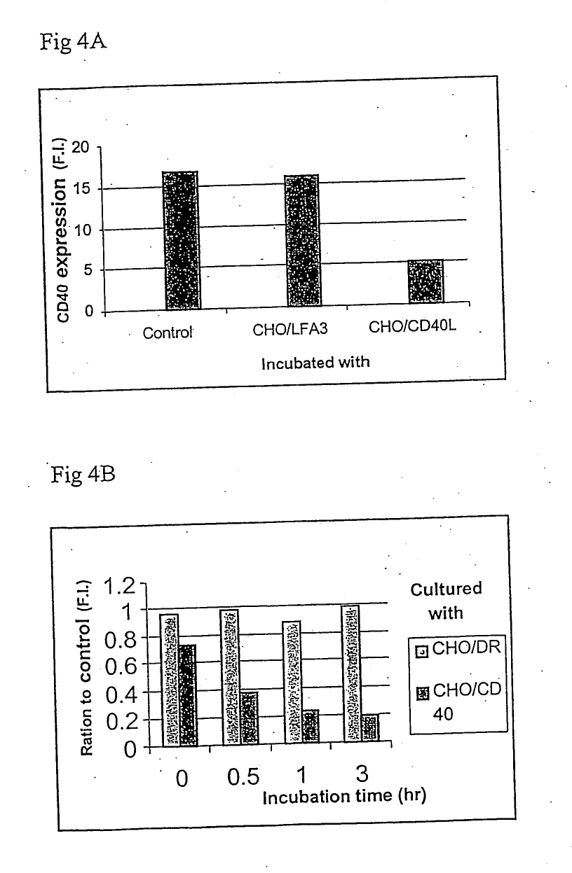

CD40L/CD40 pathway is another co-stimulation pathway for both T and B cell activation and is involved in human autoimmune diseases. The human B cell line, Ramos 2G6 4C10 cells were incubated with CHO/CD40L or control transfectants for 2 hours and then the CD40 expression was analysed with FACS. After incubation, down-modulation of CD40 was observed (FIG. 4A), together with enhanced surface expression of other adhesion molecules, e.g. CD80, CD86, LFA3, ICAM-1.

On the other hand CD40L expression on Jurkat cells was greatly reduced after incubation with CHO/CD40 for different time. After incubation expression of CD40L was analysed with FACS. The results in FIG. 4B showed that one hour after co-incubation the expression of CD40L on Jurkat cells was reduced to about 80%.

### EXAMPLE 4

Ligand Induced Receptor Down-modulation on Primary Cells

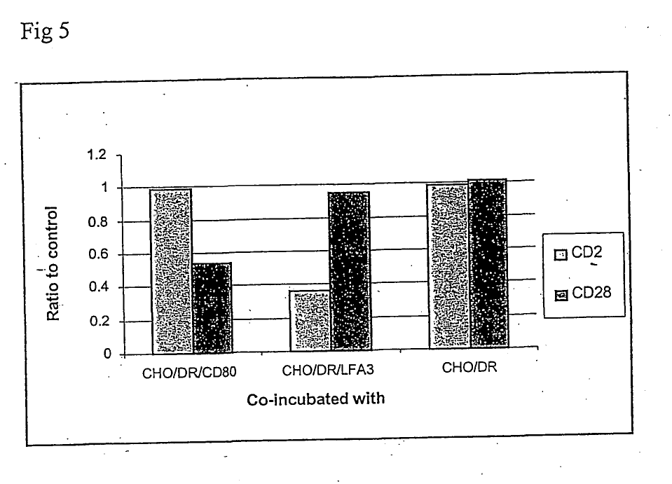

We have been using human cell lines to investigate the receptor modulation. SEA-stimulated human peripheral blood mononuclear cells (PBMC) were co-incubated with CHO cells transfected with different human molecules for 30 minutes. The cells were stained with αCD2-PE or αCD28-PE and analysed with FACS. The results from FIG. 5 show that the receptors on human primary T cells also were down-modulated by the ligands. The cultures of human PBMC were maintained by superantigen SEA. After 2-3 weeks, more than 99% of the cells. were CD3⁺CD8⁺, and CD2⁺CD28⁺. When these cells were cultured with CHO/DR transfectants, expression of either CD2 nor CD28 was influenced (FIG. 5). The expression of CD2 was down-modulated in the cultures with CHO/LFA3 transfectants and CD28 expression was down-modulated by exposure of the cells to CHO/CD80. No cross response was observed, the specificity shown already in the Jurkat/CHO system.

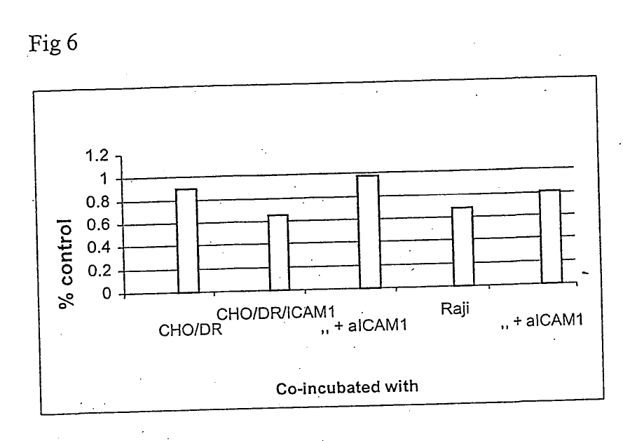

Human SEA-stimulated PBMC were then co-incubated with CHO transfectants or Raji cells, human B cell line expressing CD54, for 30 minutes. Parts of the CHO cells and Raji cells were pre-treated with αICAM-1 monoclonal antibody for 30 minutes at 4° C. and washed twice. After co-incubation the cells were stained with αCD11α-PE and analysed for CD11α expression. FIG. 6 shows that human CD11α (LFA-1) on SEA-stimulated T cells was moderately down-modulated by the ligand CD54 (ICAM-1)-transfected CHO cells. Raji cells were also inducing CD11α down-modulation. Both cell lines lost the ability after they were pretreated with αCD54 mAb, a strong evidence that the ligation of CD11α and CD54 induces CD11αdown-modulation. As far as we know we are the first to report that CD54 induces CD11α down-modulation in human.

### EXAMPLE 5

### Blockage of the Receptor Down-modulation

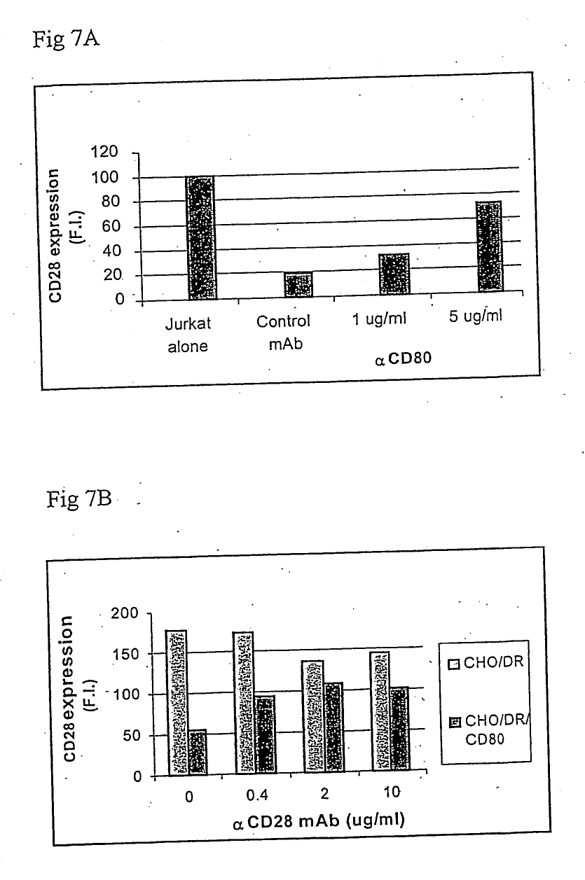

We have shown that interaction of receptors and ligands leads to internalisation of the receptor, a prerequisite for transduction of activation signals. Theoretically, a reagent which is able to block the interaction will naturally block the ligand-induced receptor internalisation. In this experiment we show that mAbs which recognise either the receptor CD28 or the ligand CD80 are able to inhibit the LIRI (FIG. 7).

CHO/CD80 transfectants were incubated with αCD80 mAb or control mAb for 30 minutes at 4° C. The cells were washed 2 times and co-incubated with Jurkat cells for 30 minutes at 37° C., The expression of CD28 receptor was analysed with FACS.

Jurkat cells were then incubated with αCD28 mAb for 30 minutes at 4° C. After 2 washes the Jurkat cells were co-incubated with CHO/DR or CHO/CD80 for 30 minutes at 37° C. The cells were incubated with saturated αCD28 mAb concentration (10 μg/ml) again for 30 minutes at 4° C. After wash the cells were stained with rabbit anti-mouse Ig conjugated with FITC. Expression of total CD28 receptors was analysed with FACS.

When the CHO/CD80 were treated with αCD80 (FIG. 7A) or the Jurkat cells were pretreated with αCD28 mnb (FIG. 7B), the receptor internalisation was inhibited. In other words, more receptors retained on the Jurkat cells. Control mAbs did not show any effects.

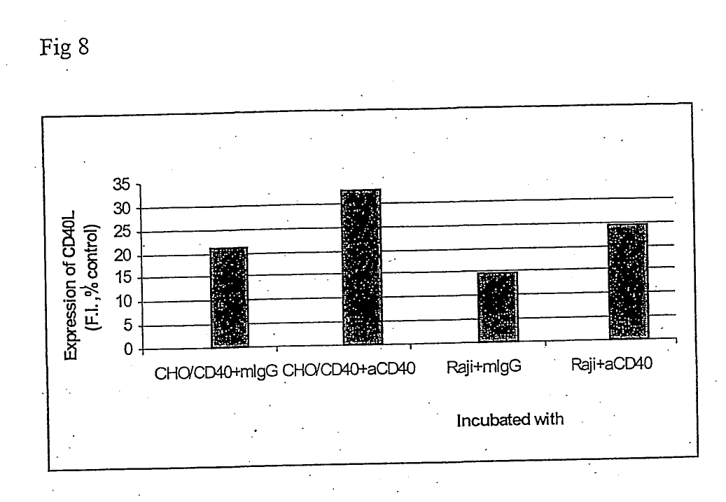

CHO/CD40 and Raji cells were pretreated with murine anti-human CD40 mAb (5 μg/ml) or control mIgG for 30 minutes. CD40L⁺Jurkat cells were then incubated with the pretreated cells for 30 minutes. The expression of CD40L on CD40L⁺Jurekat cells was analysed with FACS. Pretreatment of CHO/CD40 or Raji cells with αCD40 mAb decreased the ability of CD40 to induce CD40L down-modulation. (FIG. 8).

### EXAMPLE 6

### Blockage of the T Cell Activation

When cultured with CHO/DR/CD80 cells, in the presence of SEA, Jurkat cells will be fully activated to produce IL-2, one of the most important T cell growth factors. In this culture system SEA binds to HLA-DR molecules and a complex of SEA-DR is formed on the CHO/DR/CD80 cell surface. CD3/TCR on the surface of Jurkat cells recognises the complex and the interaction of the two parts constitutes the first activation signal for T cells. A co-stimulatory signal is necessary for a complete T cell activation, which is completed by the binding between CD28 (on Jurkat cells) and CD80 (on CHO/DR/CD80).

Jurkat cells pretreated with different concentrations of αCD28 mAb or control mAb were co-cultured with CHO/DR/CD80 transfectants and superantigen SEA (SnM) for 18 hours. IL-2 released in the culture supernatants was determined with ELISA.

Jurkat cells pretreated with αCD2 mAb or control mAb were co-cultured with CHO/DR/LFA3 transfectants for 18 hours. IL-2 in the supernatants was determined with ELISA.

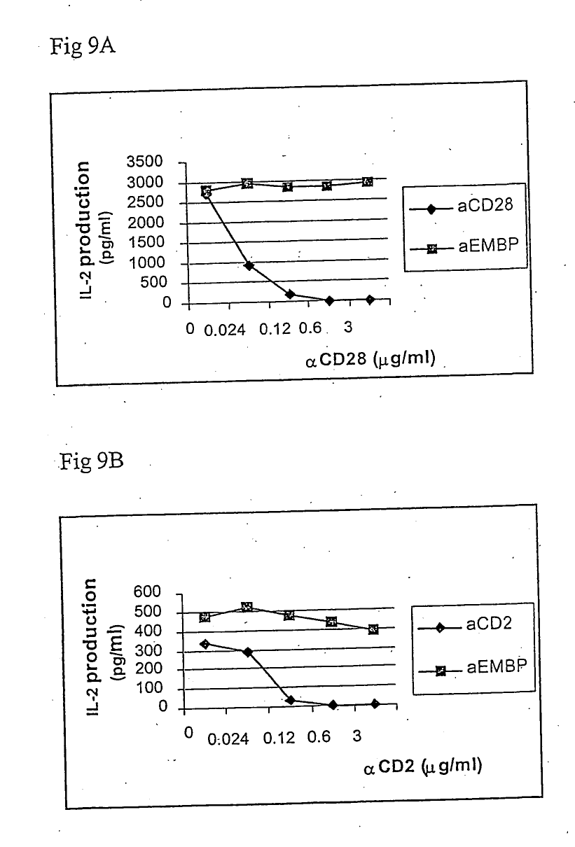

If Jurkat cells were pretreated with the mAbs, which have been shown to be able to block the ligand-induced receptor internalisation, the T cell activation was impaired. The αCD28 mAbs totally abolished IL-2 production in the Jurkat-CHO/DR/CD80 system and the αCD2 mAb also dose-dependently inhibited T cell activation in the Jurkat-CHO/DR/LFA3 system. A control mAb (αEMBP) did not interfere with the T cell activation in either system (FIG. 9). The mAbs against the ligands, CD80 or LFA3, also decreased the IL-2 production, but in a less potent manner.

### EXAMPLE 7

### Co-stimulatory Receptor Internalisation after Interaction with Ligands

Down-modulation of surface receptors on human lymphocytes induced by the ligands is a complex process and the mechanisms implied are not fully elucidated. In this experiment we show that CD2 and CD40L were internalised after the interaction with the ligand.

Jurkat cells were cultured with either CHO/LFA3 or CHO/CD40 for 1 hour. After culture parts of the cells were fixed with 4% PFA, permeabilised with 0.1% saponin and stained with αCD2 or αCD40L mAbs. Percentage of receptors retained in the cells was expressed. Frequency of positive cells was expressed.

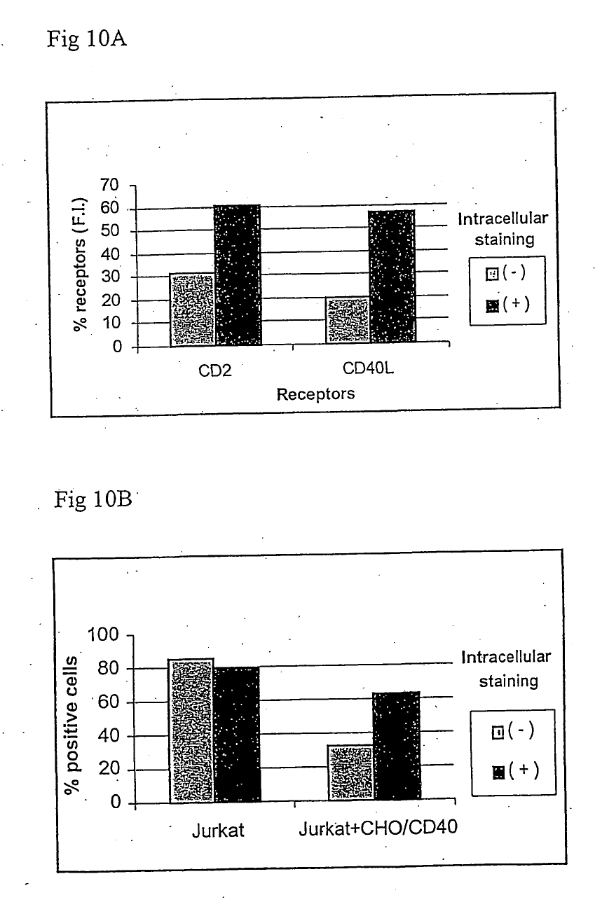

The surface expression was reduced to 70% for CD2 and 80% for CD40L after incubation with their ligands. The cells were permeabilised to allow entrance of mAbs binding to intracellular receptors. After permeabilisation CD2 and CD40L expression increased about 60% respectively, a solid evidence that the receptor internalisation (also called endocytosis) is responsible for the disappearance of surface receptors (FIG. 10A). Jurkat cells do not intracellularly express either receptor (data not shown). More than 80% of CD40L⁺-Jurkat-cells express surface CD40L and only about 30% remained positive after the ligand binding. After permeabilisation, αCD40L staining revealed that more than 60% of the cells were CD40L positive (FIG. 10B).

### EXAMPLE 8

### Substance L Blocks Ligand A Induced Receptor A Down-modulation

Substance L, a pteridine derivative with a molecular weight of 321.39,has been shown to block the binding between a receptor and its ligand from a biochemical screening program. The substance was tested in the present system, LIRI assay.

Jurkat cells were co-incubated with CHO/ligand A or CHO/ligand B transfectants for 15 minutes, with the addition of different concentrations of substance L. Expression of receptor A (in the culture with CHO/ligand A) and Receptor B (in the culture with CHO/Ligand B) was analysed with FACS.

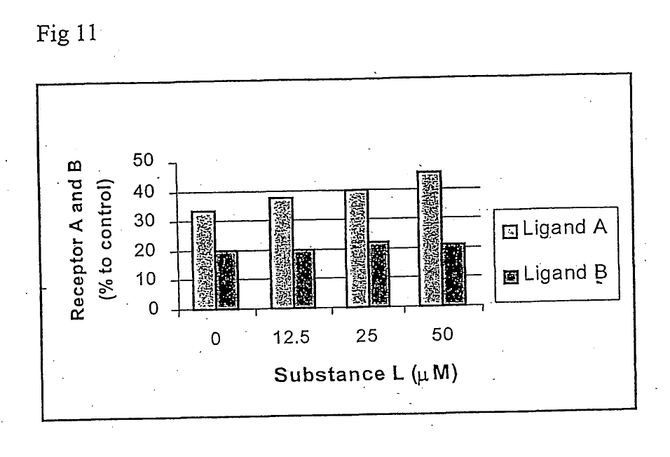

The results showed that the ligand A-induced receptor A down-modulation was dose-dependently inhibited by pretreatment of ligand A-bearing CHO cells with substance L or more receptor A retained on the cell surface after incubation with the ligand-bearing CHO cells which were pretreated with the substance. Substance L did not interfere with ligand B-induced receptor B down-modulation (FIG. 11).

### EXAMPLE 9

### Human CD80 Induces CTLA-4 Down-modulation

Materials and Methods

Cell cultures

Human peripheral blood mononuclear cells (PBMC) were isolated from the buffy coats and cultured with RPMI 1640 supplemented with 10% fetal calf sera and SEE (5 nM) for 72 hours. To study modulation of CTLA-4 by the ligand CD80, the SEE-activated cells were incubated with CHO/C80 transfectants for 30 minutes at a ratio of 5:1 at 37° C. The cells were washed and stained with PE-conjugated αCTLA-4 (PharMingen, Cat. No. 555853) and analyzed with FACS.

Results

Down -modulation of CTLA-4 by CD80

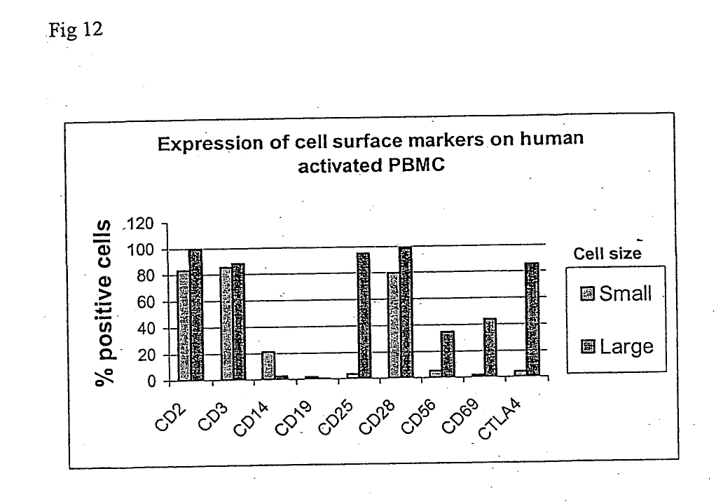

Human resting T cells do not express CTLA-4 on the cell surface (data not shown). After activation with SEE human PBMC could be divided-into 2 groups according to cell size. Results from FIG. 12 show that as high as 80% of large cells express CTLA-4 along with elevated expression of other T activation makers (CD25, CD56, CD69). Small cells remained similar phenotype as resting PBMC.

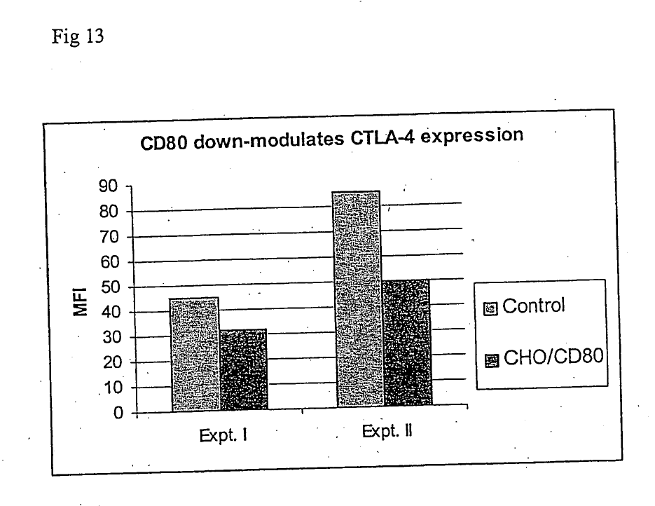

After activation the PBMC cells were mixed with CHO/CD80 cells and incubated for another 30 minutes. Modulation of CTLA-4 was analyzed FACS. Results in FIG. 13 show that CTLA-4 expression was down-modulated by CD80. Results from parallel cultures showed that CD28 and CD2 were also down-modulated respectively by CD80 and LFA₃ transfectants and the LFA₃ was not able to down-modulate CTLA-4 expression (data not shown). These results demonstrate that the inducible human T cell co-stimulatory receptor, CTLA-4, were also down-modulated by interacting with the counterreceptor CD80 and a strict specificity also exists in the modulation of CTLA-4.

References

1. Tivol, A E et al, “Co-stimulation and autoimmunity”, Current Opinion in Immunology, 8, pp 822-830 (1996).

2. June, C H et al, “Role of the CD28 receptor in T cell activation”, Immunology Today, 11, pp 211-216 (1990).

3. Wingren, A G et al, “T cell activation pathways: B7, LFA-3, and ICAM-1 shape unique T cell profiles”, Crit Rev Immunol, 15, pp 235-253 (1995).

4. Melero, I et al, “Monoclonal antibodies against 4-1 BB T cell activation molecule eradicate established tumors”, Nature Medicine, 6 pp 682-685 (1997).

5. Marsh, M et al, “The structural era of endocytosis”, Science, 285, pp 215-220 (July 1999).

6. Eck, S C et al, “Differential down-regulation of CD28 by B7-1 and B7-2 engagement”, Transplantation, 64, pp 1497-1499 (Nov 1997).

1. A method of screening compounds for their ability of inhibiting ligand-induced co-stimulatory receptor internalisation pathways in immune competent human cells, characterised by
incubating said immune competent cells at conditions capable of inducing co-stimulatory receptor internalisation in the presence of at least one test compound; and
determining the suppression of the ligand-induced co-stimulatory receptor internalisation.

2. A method according to claim 1, whereby said immune competent cells are leukocytes.

3. A method according to claim 2, whereby said leukocytes are lymphocytes.

4. A method according to claim 3, whereby said lymphocytes are T-cells.

5. A method according to claim 4, whereby said T-cells are Jurkat cells.

6. A method according to claim 2, whereby said leukocytes are antigen presenting cells.

7. A method according to claim 6, whereby said antigen presenting cells are B-cells.

8. A method according to any one of claims 1-7, whereby said conditions imply culturing the immune competent cells with Chinese hamster ovarian (CHO) cells transfected with a DNA, which codes for at least one human ligand.

9. A method according to claim 8, whereby the CHO cells are transfected with a DNA encoding at least one human ligand or receptor chosen from the group comprising ICAM, CD54(LFA3), CD40, CD80, CD86, CD154(CD40L).

10. A method according to any one of claims 1-9, whereby the test compound is a low molecular weight compound.

11. A method according to claim 10, whereby the test compound has a molecular weight of up to about 500.

12. A method according to any one of claims 1-11, whereby said determination of the suppression is made by flow cytometry or confocal microscopy analysis of the cells.

13. A method according to any one of claims 1-12, which is automated for high content screening (HCS) or medium through-put screening (MTS).

14. A method according to any one of claims 1-13, whereby said pathways are chosen from the group of receptor-ligand pairs comprising CD40/CD154 (CD40L); CD2/LFA3; CD28/CD80, CD86; and CD11/ICAM.

15. A kit for use in screening compounds for their ability of inhibiting ligand-induced co-stimulatory receptor internalisation in immune competent human cells, comprising
means for culturing immune competent human cells;
means for inducing co-stimulatory-receptor internalisation;
means for incubating the immune competent human cells with at least one test compound;
means for marking the receptors; and
means for determining suppression of the ligand-induced co-stimulatory receptor internalisation.

16. A kit according to claim 15, whereby a conjugate with an isotope or a fluorescent protein is used for marking the receptors.

17. A kit according to claims 15 or 16, whereby flow cytometry or confogal microscopy is used for determining suppression of the ligand-induced co-stimulatory receptor internalisation.

18. An immuno-regulatory drug, capable of blocking down-modulation of a ligand-induced receptor thus preventing ligand-induced receptor internalisation.

19. An immuno-regulatory drug according to claim 18, which is a low molecular weight compound.

20. An immuno-regulatory drug according to claim 18 or 19, which is a compound having a molecular weight of up to about 500.
